(19) United States
(12) Patent Application Publication
(10) Pub. No.: US 2005/0003387 A1
(43) Pub. Date: Jan. 6, 2005

(54) Methods and compositions for modulating apoptosis

(75) Inventors: Pedro Aza-Blanc, San Diego, CA (US); Michael P. Cooke, San Diego, CA (US); Quinn L. Deveraux, San Diego, CA (US); Christopher L. Cooper, San Diego, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, Hamilton (BM)

(21) Appl. No.: 10/783,391

(22) Filed: Feb. 20, 2004

Related U.S. Application Data
(60) Provisional application No. 60/448,960, filed on Feb. 21, 2003.
(60) Provisional application No. 60/494,527, filed on Aug. 12, 2003.

(51) Int. Cl. C12Q 1/68; G01N 33/53; G01N 33/567; G01N 33/574

(52) U.S. Cl. 435/6; 435/7.23

(57) ABSTRACT

The invention provides novel modulatory polypeptides of TRAIL-induced apoptosis. The invention also provides methods for screening modulators of TRAIL-induced apoptosis. The methods comprise first screening test agents for modulators of a novel modulatory polypeptide of TRAIL-induced apoptosis and then further screening the identified modulating agents for modulators of TRAIL-induced apoptosis. The invention further provides methods and pharmaceutical compositions for modulating apoptosis of cells and for treating diseases and conditions such as cancers.

### CROSS-REFERENCE TO RELATED APPLICATIONS

This application claims the benefit of priority to U.S. Provisional Patent Application Ser. Nos. 60/448,960 (filed Feb. 21, 2003) and 60/494,527 (filed Aug. 12, 2003). The disclosures of these earlier filed patent applications are incorporated herein by reference in their entirety and for all purposes.

### FIELD OF THE INVENTION

The present invention generally relates to methods for identifying modulators of TRAIL-induced apoptosis and therapeutic applications of such modulators.

### BACKGROUND OF THE INVENTION

Apoptosis is a highly conserved cell suicide program essential for development and tissue homeostasis of all metazoan organisms. Changes to the apoptotic pathway that prevent or delay normal cell turnover can be just as important in the pathogenesis of diseases as are abnormalities in the regulation of the cell cycle. Like cell division, which is controlled through complex interactions between cell cycle regulatory proteins, apoptosis is similarly regulated under normal circumstances by the interaction of gene products that either prevent or induce cell death.

TNF-related apoptosis-inducing ligand (TRAIL, also referred to as Apo2L) is a widely expressed member of the tumor necrosis factor (TNF) superfamily. Experimentally, recombinant TRAIL protein was shown to induce apoptosis in a variety of tumor cells, while leaving normal cells intact. Binding of TRAIL to death receptors DR4 and DR5 induces apoptosis through recruitment of the adapter molecule FADD and pro-caspase-8, which form the death-inducing signaling complex (DISC) where pro-caspase-8 is activated. Depending on the cell type, active Caspase-8 can directly lead to the activation of downstream effector caspases such as Caspase-3 (type-1-cells). In type-1′-cells, this death receptor or extrinsic pathway engages the so called intrinsic pathway by Caspase-8-mediated cleavage of the pro-apoptotic BCL-2 family member BID, which promotes the mitochondrial release of cytochrome c and SMAC. Once released into the cytoplasm, cytochrome c associates with APAF-1 and pro-caspase-9 forming a complex called the “apoptosome”, which leads to the activation of pro-caspase-9 and effector caspases such as Caspase-3. SMAC binds to members of the inhibitor of apoptosis (IAP) protein family, such as XIAP, and thereby prevents XIAP mediated inhibition of Caspase-3, -7 and -9. See, e.g., Pan et al, Science 277:815-8 (1997); Sheridan, et al., Science 277:818-21 (1997); Walczak et al, EMBO J. 16:5386-97 (1997); Deveraux and Reed, Genes Dev 13, 239-52 (1999); and Verhagen and Vaux, Apoptosis 7, 163-6 (2002).

There is a need in the art for better means for modulating apoptosis and for treating cancer. The present invention addresses this and other needs.

### SUMMARY OF THE INVENTION

In one aspect, the present invention provides methods for identifying novel modulators of TRAIL-induced apoptosis. The methods comprise (a) assaying a biological activity of a polypeptide modulator of TRAIL-induced apoptosis identified herein, or a fragment of said polypeptide, in the presence of test agents to identify one or more modulating agents that modulate the biological activity, and (b) testing one or more of the modulating agents for ability to modulate TRAIL-induced apoptosis.

In some methods, the polypeptide modulator enhances TRAIL-induced apoptosis and is encoded by a gene selected from the group consisting of DOBI, Gsk3a, and SRP72. In some of these methods, the polypeptide modulator is Gsk3a, and the biological activity is its kinase activity. In some methods, the polypeptide modulator is SRP72, and the biological activity is facilitating protein translocation.

In some other methods, the polypeptide modulator inhibits TRAIL-induced apoptosis and is encoded by a gene selected from the group consisting of MIRSA, JIK, and PLXNB1. In some of these methods, the polypeptide modulator is JIK, and the biological activity is its kinase activity. In some methods, wherein the polypeptide modulator is PLXNB1, and the biological activity is PLXNB1 binding to semaphorin.

In some of the methods, (a) comprises testing the test agents for ability to bind to the polypeptide modulator. In some methods, (a) comprises testing the test agents for ability to modulate cellular level of the polypeptide modulator. In some methods, (b) comprises testing the modulating agents for ability to modulate caspase activity. In some methods, the assaying of the biological activity of the polypeptide modulator occurs in a cell.

In another aspect, the invention provides methods for modulating TRAIL-induced apoptosis activity of a cell. These methods comprise contacting the cell with an effective amount of a novel modulator of TRAIL-induced apoptosis that is identified in accordance with methods of the invention. In some of these methods, the modulator enhances TRAIL-induced apoptosis activity. Some of the methods are directed to tumor cells. In some methods, the cell is present in a subject. In some of the methods, the subject is also administered a pharmaceutical composition comprising an effective amount of a TRAIL polypeptide or a fragment thereof.

In another aspect, the present invention provides methods treating cancer in a subject. The methods comprise promoting TRAIL-induced apoptosis in the subject by administering to the subject a pharmaceutical composition comprising an effective amount of a novel modulator of TRAIL-induced apoptosis that is identified in accordance with methods of the invention. In some of these methods, the pharmaceutical composition further comprises an effective amount of a TRAIL polypeptide or a fragment thereof.

A further understanding of the nature and advantages of the present invention may be realized by reference to the remaining portions of the specification and claims.

### BRIEF DESCRIPTION OF THE DRAWINGS

FIGS. 1A-1C show siRNA-based screen for Trail sensitivity. (A) Screening strategy. siRNA duplexes were spotted onto 384 well plates in duplicate and HeLa cells were reverse transfected onto the wells. Cells were incubated for 48 hours to allow target decay, and treated with or without TRAIL. Viability was measured 20 hours after TRAIL treatment using alamar blue. A sensitivity ratio was determined for each siRNA for comparison with a total of 60 values obtained with control siRNAs; (B) viability distributions from alamar blue readings after transfecting the siRNA collection in the absence (lines corresponding to the right two peaks) or presence (lines corresponding to the left two peaks) of TRAIL compared to controls (hatched lines). Data correspond to average values obtained from 2 screens done in parallel; (C) Histogram showing distribution sensitivity ratios derived from 2 parallel experiments across the siRNA collection and compared to controls (siRNA collection, blue line. Controls, blue hatched line).

FIGS. 2A-2C show confirmation of selected genes detected in the screen. Two additional siRNAs with independent sequence (siRNA“1” and siRNA“2”) were designed for each target and used to confirm that the screen results are due to target inhibition. siRNA control used was siGL2. Experiments were performed in normal serum conditions. (A) Effect of selected inhibitor siRNAs on TRAIL-dependent caspase activation. Columns marked “−” indicate no TRAIL treatment; and columns marked “+” indicate treatment with 1 μg/ml TRAIL. Selected targets were GSK3α, the uncharacterized FLJ32312, and the signal recognition particle component SRP72. Performance of siRNAs against GSK3β is also shown. Values are normalized to caspase activity detected for control (siGL2) in the presence of TRAIL (=3). GSK3β siRNAs behaved as control siRNA and did not prevent caspase activation; (B) Western analysis of GSK3α and GSK3β levels after transfection of GSK3α and GSK3β siRNAs 1 and 2. The GSK3α siRNA present in the screen (GSK3αS) is also included. All of them were efficient inhibitors of their respective targets; (C) Effect of selected enhancer siRNAs on caspase activation by TRAIL. Selected targets were the semaphorin receptor PLXNB1, JNK inhibitory kinase (JIK), and the uncharacterized gene FLJ21802. PAK1 was also included in the study as a kinase with known anti-apoptotic activity. Effects on caspase activity in the absence of TRAIL (“−” columns) or under 100 ng/ml TRAIL treatment (“+” columns) are normalized as in (B).

FIGS. 3A-3B show biochemical mapping of inhibitory hits (rate limiting activities of TRAIL induced apoptosis). (A) siRNAs against SRP72, GSK3α and DOBI were transfected in parallel with a negative control (sigl2) and 2 positive controls (siCASP8 and siBID). 48 h later cells were treated with or without TRAIL (1 μg/ml) and western analysis was performed with antibodies to detect Caspase 8, BID, Caspase 9, Caspase 3 cleavage. SRP72 is required for Caspase 8 activation by TRAIL signaling. GSK3α showed a similar though weaker activity and might be acting at other levels. STIA blocked Caspase 9 activation despite it did not prevent Bid or Caspase 3 cleavage; (B) Schematic representation of the pathway and positions at which interaction of these genes is detected.

### DETAILED DESCRIPTION

The present invention is predicated in part on the discovery by the present inventors of a number of genes that modulate TRAIL induced apoptosis. Using siRNA-based loss of function screening as a mammalian genetics tool, the present inventors discovered a number of genes that impact TRAIL-induced apoptosis. In accordance with these discoveries, the present invention provides novel modulators of TRAIL-induced apoptosis and methods for identifying such modulators. The invention also provides methods for modulating TRAIL-induced apoptosis and for treating various tumors and diseases or conditions, e.g., by promoting cell death, in a subject. The following sections provide guidance for making and using the compositions of the invention, and for carrying out the methods of the invention.

I. Definitions

Unless defined otherwise, all technical and scientific terms used herein have the same meaning as commonly understood by those of ordinary skill in the art to which this invention pertains. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); and Hale & Marham, THE HARPER COLLINS DICTIONARY OF BIOLOGY (1991). In addition, the following definitions are provided to assist the reader in the practice of the invention.

The term “agent” or “test agent” includes any substance, molecule, element, compound, entity, or a combination thereof. It includes, but is not limited to, e.g., protein, polypeptide, small organic molecule, polysaccharide, polynucleotide, and the like. It can be a natural product, a synthetic compound, or a chemical compound, or a combination of two or more substances. Unless otherwise specified, the terms “agent”, “substance”, and “compound” can be used interchangeably.

The term “analog” is used herein to refer to a molecule that structurally resembles a reference molecule but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs, to identify variants of known compounds having improved traits (such as higher binding affinity for a target molecule) is an approach that is well known in pharmaceutical chemistry.

As used herein, “contacting” has its normal meaning and refers to combining two or more agents (e.g., polypeptides or small molecule compounds) or combining agents and cells (e.g., a polypeptide and a cell). Contacting can occur in vitro, e.g., combining two or more agents or combining a test agent and a cell or a cell lysate in a test tube or other container. Contacting can also occur in a cell or in situ, e.g., contacting two polypeptides in a cell by coexpression in the cell of recombinant polynucleotides encoding the two polypeptides, or in a cell lysate.

A “host cell,” as used herein, refers to a prokaryotic or eukaryotic cell that contains heterologous DNA that has been introduced into the cell by any means, e.g., electroporation, calcium phosphate precipitation, microinjection, transformation, viral infection, and/or the like.

The terms “identical”, “sequence identical” or “sequence identity” in the context of two nucleic acid sequences or amino acid sequences refers to the residues in the two sequences which are the same when aligned for maximum correspondence over a specified comparison window. A “comparison window”, as used herein, refers to a segment of at least about 20 contiguous positions, usually about 50 to about 200, more usually about 100 to about 150 in which a sequence may be compared to a reference sequence of the same number of contiguous positions after the two sequences are aligned optimally. Methods of alignment of sequences for comparison are well-known in the art. Optimal alignment of sequences for comparison may be conducted by the local homology algorithm of Smith and Waterman (1981) Adv. Appl. Math. 2:482; by the alignment algorithm of Needleman and Wunsch (1970) J. Mol. Biol. 48:443; by the search for similarity method of Pearson and Lipman (1988) Proc. Nat. Acad. Sci U.S.A. 85:2444; by computerized implementations of these algorithms (including, but not limited to CLUSTAL in the PC/Gene program by Intelligentics, Mountain View, Calif.; and GAP, BESTFIT, BLAST, FASTA, or TFASTA in the Wisconsin Genetics Software Package, Genetics Computer Group (GCG), 575 Science Dr., Madison, Wis., U.S.A.). The CLUSTAL program is well described by Higgins and Sharp (1988) Gene 73:237-244; Higgins and Sharp (1989) CABIOS 5:151-153; Corpet et al. (1988) Nucleic Acids Res. 16:10881-10890; Huang et al (1992) Computer Applications in the Biosciences 8:155-165; and Pearson et al. (1994) Methods in Molecular Biology 24:307-331. Alignment is also often performed by inspection and manual alignment. In one class of embodiments, the polypeptides herein are at least 70%, generally at least 75%, optionally at least 80%, 85%, 90%, 95% or 99% or more identical to a reference polypeptide, e.g., a TRAIL-modulatory polypeptide encoded by a polynucleotide in Tables 1 and 2, e.g., as measured by BLASTP (or CLUSTAL, or any other available alignment software) using default parameters. Similarly, nucleic acids can also be described with reference to a starting nucleic acid, e.g., they can be 50%, 60%, 70%, 75%, 80%, 85%, 90%, 95%, 99% or more identical to a reference nucleic acid, e.g., a polynucleotide in Tables 1 and 2, e.g., as measured by BLASTN (or CLUSTAL, or any other available alignment software) using default parameters.

The terms “substantially identical” nucleic acid or amino acid sequences means that a nucleic acid or amino acid sequence comprises a sequence that has at least 90% sequence identity or more, preferably at least 95%, more preferably at least 98% and most preferably at least 99%, compared to a reference sequence using the programs described above (preferably BLAST) using standard parameters. For example, the BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)). Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

The terms “mimetic” and “peptidomimetic” refer to a synthetic chemical compound that has substantially the same structural and functional characteristics of a naturally or non-naturally occurring polypeptide (e.g., SMAC). Peptide analogs are commonly used in the pharmaceutical industry as non-peptide drugs with properties analogous to those of the template peptide. These types of non-peptide compound are termed “peptide mimetics” or “peptidomimetics” (Fauchere, J. Adv. Drug Res. 15:29 (1986); Veber and Freidinger TINS p. 392(1985); and Evans et al. J. Med. Chem. 30:1229 (1987), which are incorporated herein by reference). Peptide mimetics that are structurally similar to therapeutically useful peptides may be used to produce an equivalent or enhanced therapeutic or prophylactic effect. Generally, peptidomimetics are structurally similar to a paradigm polypeptide (i.e., a polypeptide that has a biological or pharmacological activity), such as found in a polypeptide of interest, but have one or more peptide linkages optionally replaced by a linkage selected from the group consisting of, e.g., —CH2NH—, —CH2S—, —CH2—CH2—, —CH═CH— (cis and trans), —COCH2—, —CH(OH)CH2—, and —CH2SO—. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. For example, a mimetic composition is within the scope of the invention if it is capable of carrying out at least one of the binding or enzymatic activities of a polypeptide of interest.

The term “modulate” refers to a change in the cellular level or other biological activities of an apoptosis-modulatory polypeptide or a change in TRAIL-induced apoptosis activities. Modulation can be up-regulation (i.e., activation or stimulation) or down-regulation (i.e. inhibition or suppression). The mode of action of a modulator can be direct, e.g., through binding to the apoptosis-modulatory polypeptide or to genes encoding the polypeptide. The change can also be indirect, e.g., through binding to and/or modifying (e.g., enzymatically) another molecule which otherwise modulates the apoptosis-modulatory polypeptide or TRAIL-induced apoptosis.

A “variant” of a molecule such as a TRAIL-modulatory polypeptide is meant to refer to a molecule substantially similar in structure and biological activity to either the entire molecule, or to a fragment thereof. Thus, provided that two molecules possess a similar activity, they are considered variants as that term is used herein even if the composition or secondary, tertiary, or quaternary structure of one of the molecules is not identical to that found in the other, or if the sequence of amino acid residues is not identical.

II. Genes Encoding Novel Modulators of TRAIL-Induced Apoptosis

The present inventors identified a number of cellular components that modulate the apoptotic response to TRAIL. As detailed in the Examples below, HeLa cells were screened using an siRNA library directed against 510 genes including most known and predicted kinases. This siRNA-mediated pathway mapping revealed that the genes shown in Tables 1 and 2 are potential apoptosis inhibitors and enhancers, respectively. Polypeptides encoded by these genes are termed herein “polypeptide modulators of TRAIL-induced apoptosis,” “TRAIL-modulatory polypeptides” or “apoptosis-modulatory polypeptides.” They display a spectrum of activities that either limit or enhance cell sensitivity to TRAIL.

A few of these identified genes were further examined to confirm their activities on TRAIL-induced apoptosis, including hypothetical protein FLJ32312 (DOBI), Gsk3α, SRP72, hypothetical protein FLJ21802 (MIRSA, “mina related suppressor of apoptosis”), JIK, and PLXNB1. It was discovered that GSK3α, SRP72 and the novel gene DOBI are required at distinct steps in the apoptotic cascade. Additionally, several genes including JIK, PLXNB1, and MIRSA were shown to prevent TRAIL-mediated apoptosis. Further, it was discovered that siRNAs targeting to the genes that inhibit TRAIL-induced apoptosis (other than siRNAs targeting PLXNB1) also induced an increase in TRAIL-independent caspase activation, supporting a more general anti-apoptotic role for these genes.

The hypothetical protein FLJ32312, designated DOBI by the present inventors, is encoded by polynucleotide sequences with GenBank accession numbers AK056874.1, NM_{—}144709.1, AK021502.1, and AL832208.1. Human Gsk3α (glycogen synthase kinase-α) amino acid acid sequence is disclosed in the art (accession numbers NP_{—}063937 and AAH51865). GenBank accession numbers for the corresponding polynucleotide sequences are NM_{—}019884.1 and BC051865. This enzyme is a serine-thereonine kinase and is involved in the regulation of a variety of cellular processes and also implicated in the pathogenesis of several human diseases (see, e.g., Cohen et al., Nat Rev Mol Cell Biol. 2(10):769-76, 2001 and Dominguez et al., Dev Biol. 235(2):303-13, 2001). Proteins phosphorylated by this enzyme include eukaryotic initiation factor (eIF-2B), glycogen synthase, and β-catenin. SRP72 is a 72 kDa component of a ribonucleoprotein, the signal recognition particle (SRP). SRP is composed of an Alu domain and an S domain. The S domain contains unique sequence SRP RNA and four SRP proteins: SRP19, SRP54, SRP68, and SRP72 (Politz et al., Proc Natl Acad Sci USA 97(1):55-60, 2000). Human SRP72 polunlcoeitde and amino acid sequences are known in the art (accession numbers NM_{—}006947 and NP_{—}008878, respectively). SRP functions to recognize the signal peptide of nascent transcripts, attach the translating ribosome to the endoplasmic reticulum (ER), and facilitate translocation into the ER lumen. SRP72 is essential for protein translocation.

The hypothetical protein FLJ21802, designated MIRSA by the present inventors, is encoded by polynucleotide sequences with GenBank accession numbers AK025455.1, NM_{—}024644.1, and BC011350.1. JIK is a serine/threonine kinase that inhibits the c-Jun N-terminal kinase (JNK) cascade (Tassi et al., J Biol Chem 274: 33287-95, 1999). Polynucleotide sequences (accession numbers NM_{—}016281 and AF179867) and amino acid sequences (NP_{—}057365 and AAF14559) encoding human JIK are known in the art. PLXNB1 encodes Plexin-B 1, a high-affinity receptor for semaphorin CD100 (Sema4D). Plexin-B1 is expressed by bone marrow stromal cells, follicular dendritic cells, and activated T lymphocytes. It promotes survival in certain situations (Aurandt et al., Proc Natl Acad Sci USA 99, 12085-90, 2002; and Granziero et al., Blood 101(5):1962-9 2003). Polynucleotide and amino acid sequences of PLXNB1 are known in the art (accession numbers NM_{—}002673 and NP_{—}002664, respectively).

TABLE 1
Apoptosis-enhancing genes identified by siRNA targeting

| Accession No. | Symbol | SR (%) | P value |
|---|---|---|---|
| NM_006947 | SRP72 | 94 | 8.5E−22 |
| NM_001715 | BLK | 79 | 5.9E−16 |
| XM_086132 | PKM2 like | 75 | 3.6E−14 |
| NM_019884 | GSK3A | 73 | 3.6E−14 |
| NM_144709 | FLJ32312 | 72 | 1.8E−12 |
| NM_002467 | C-MYC | 69 | 5.3E−11 |
| NM_025133 | FLJ12673 | 65 | 2.9E−09 |
| NM_002944 | ROS1 | 61 | 2.5E−08 |
| NM_005158 | ABL2 | 61 | 2.9E−08 |
| NM_004705 | DAP4 | 61 | 3.2E−08 |
| NM_002753 | JNK3 | 60 | 7.8E−08 |
| NM_003199 | TCF4 | 59 | 2.0E−07 |
| NM_022575 | VPS16 | 59 | 2.1E−07 |
| NM_000858 | GUK1 | 59 | 3.3E−07 |
| NM_006257 | PRKCQ | 55 | 8.9E−06 |
| NM_006252 | PRKAA2 | 54 | 5.3E−05 |
| AK074085 | FLJ00156 | 53 | 8.6E−05 |
| NM_006254 | PRKCD | 53 | 1.0E−04 |
| NM_001569 | IRAK1 | 52 | 1.3E−04 |
| NM_004422 | DVL2 | 52 | 1.3E−04 |

TABLE 2
Apoptosis-inhibiting genes identified by siRNA targeting

| Accession No. | Symbol | SR (%) | P value |
|---|---|---|---|
| NM_012290 | TLK1 | 15 | 3.7E−21 |
| NM_016231 | NLK | 15 | 5.7E−22 |
| NM_015071 | GRAF | 14 | 1.4E−21 |
| NM_000162 | GCK | 14 | 2.5E−22 |
| NM_005163 | AKT1 | 14 | 1.1E−22 |
| NM_002749 | ERK5 | 14 | 6.8E−23 |
| NM_002350 | LYN | 14 | 3.0E−24 |
| NM_004755 | RPS6KA5 | 13 | 5.6E−24 |
| NM_004336 | BUB1 | 13 | 8.2E−27 |
| NM_005592 | MUSK | 12 | 3.2E−27 |
| NM_024644 | FLJ21802 | 12 | 9.5E−28 |
| NM_005248 | FGR | 12 | 2.2E−28 |
| NM_000020 | ACVRL1 | 12 | 3.4E−28 |
| NM_002757 | MEKK5 | 11 | 1.4E−28 |
| XM_047620 | PIP5K1C | 11 | 2.8E−33 |
| NM_004759 | MAPKAPK2 | 10 | 1.9E−18 |
| NM_052859 | RFT1 | 10 | 7.8E−35 |
| NM_003684 | MKNK1 | 10 | 9.3E−37 |
| NM_016281 | JIK | 9 | 4.4E−37 |
| NM_002673 | PLXNB1 | 8 | 2.7E−38 |

III. Methods for Screening Modulators of TRAIL-Induced Apoptosis
A. General Scheme and Assay Systems

The apoptosis-modulatory polypeptides described above provide novel targets for screening modulators (agonists or antagonists) of the TRAIL-induced apoptosis. Employing these novel targets, the present invention provides methods for screening agents or compounds that modulate activities of the TRAIL-induced apoptosis. Various biochemical and molecular biology techniques well known in the art can be employed to practice the present invention. Such techniques are described in, e.g., Sambrook et al., Molecular Cloning: A Laboratory Manual, Cold Spring Harbor Press, N.Y., Second (1989) and Third (2000) Editions; and Ausubel et al., Current Protocols in Molecular Biology, John Wiley & Sons, Inc., New York (1987-1999).

In some methods, test agents are first assayed for their ability to modulate a biological activity of an apoptosis-modulatory polypeptide (“the first assay step”). Modulating agents thus identified are then subject to further screening for ability to modulate TRAIL-induced apoptosis activities, typically in the presence of the apoptosis-modulatory polypeptide (“the second testing step”). Depending on the apoptosis-modulatory polypeptide employed in the method, modulation of different biological activities of the apoptosis-modulatory polypeptide can be assayed in the first step. For example, a test agent can be assayed for binding to the apoptosis-modulatory polypeptide. The test agent can be assayed for activity to modulate expression level of the apoptosis-modulatory polypeptide, e.g., transcription or translation. The test agent can also be assayed for activities in modulating cellular level or stability of the apoptosis-modulatory polypeptide, e.g., post-translational modification or proteolysis.

If the apoptosis-modulatory polypeptide has a known or well established biological or enzymatic function (e.g., kinase activity of Gsk3α and JIK), the biological activity monitored in the first screening step can be the specific biochemical or enzymatic activity of the apoptosis-modulatory polypeptide. In an exemplary embodiment, the apoptosis-modulatory polypeptide is a kinase and test agents are first screened for modulating the kinase's activity in phosphorylating a substrate. The substrate can be a polypeptide known to be phosphorylated by the kinase (e.g., β-catenin for GSK3α). Once test agents that modulate the apoptosis-modulatory polypeptides are identified, they are typically further tested for ability to modulate the TRAIL-induced apoptosis. For example, the test agents can be further tested for ability to modulate caspase activity in the presence of TRAIL, as detailed in the Examples below.

If a test agent identified in the first screening step modulates cellular level (e.g., by altering transcription activity) of the apoptosis-modulatory polypeptide, it would indirectly modulate the TRAIL-induced apoptosis. On the other hand, if a test agent modulates an activity other than cellular level of the apoptosis-modulatory polypeptide, then the further testing step is needed to confirm that their modulatory effect on the apoptosis-modulatory polypeptide will indeed lead to modulation of TRAIL-induced apoptosis. For example, a test agent which modulates kinase activity of an apoptosis-modulatoty polypeptide needs to be further tested in order to confirm that modulation of the kinase activity can result in modulation of TRAIL-induced apoptosis.

In both the first assaying step and the second testing step, either an intact apoptosis-modulatory polypeptide, or its fragments, analogs, or functional derivatives can be used. The fragments that can be employed in these assays usually retain one or more of the biological activities of the apoptosis-modulatory polypeptide (e.g., kinase activity if the apoptosis-modulatory employed in the first assaying step is a kinase). Fusion proteins containing such fragments or analogs can also be used for the screening of test agents. Functional derivatives of apoptosis-modulatory polypeptides and TRAILs usually have amino acid deletions and/or insertions and/or substitutions while maintaining one or more of the bioactivities and therefore can also be used in practicing the screening methods of the present invention. A functional derivative of an apoptosis-modulatory polypeptide can be prepared from a naturally occurring or recombinantly expressed protein by proteolytic cleavage followed by conventional purification procedures known to those skilled in the art. Alternatively, the functional derivative can be produced by recombinant DNA technology by expressing only fragments of an apoptosis-modulatory polypeptide that retains one or more of their bioactivities.

B. Test Agents

Test agents that can be screened with methods of the present invention include polypeptides, beta-turn mimetics, polysaccharides, phospholipids, hormones, prostaglandins, steroids, aromatic compounds, heterocyclic compounds, benzodiazepines, oligomeric N-substituted glycines, oligocarbamates, polypeptides, saccharides, fatty acids, steroids, purines, pyrimidines, derivatives, structural analogs or combinations thereof. Some test agents are synthetic molecules, and others natural molecules.

Test agents are obtained from a wide variety of sources including libraries of synthetic or natural compounds. Combinatorial libraries can be produced for many types of compound that can be synthesized in a step-by-step fashion. Large combinatorial libraries of compounds can be constructed by the encoded synthetic libraries (ESL) method described in WO 95/12608, WO 93/06121, WO 94/08051, WO 95/35503 and WO 95/30642. Peptide libraries can also be generated by phage display methods (see, e.g., Devlin, WO 91/18980). Libraries of natural compounds in the form of bacterial, fungal, plant and animal extracts can be obtained from commercial sources or collected in the field. Known pharmacological agents can be subject to directed or random chemical modifications, such as acylation, alkylation, esterification, amidification to produce structural analogs.

Combinatorial libraries of peptides or other compounds can be fully randomized, with no sequence preferences or constants at any position. Alternatively, the library can be biased, i.e., some positions within the sequence are either held constant, or are selected from a limited number of possibilities. For example, in some cases, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, or to purines.

The test agents can be naturally occurring proteins or their fragments. Such test agents can be obtained from a natural source, e.g., a cell or tissue lysate. Libraries of polypeptide agents can also be prepared, e.g., from a cDNA library commercially available or generated with routine methods. The test agents can also be peptides, e.g., peptides of from about 5 to about 30 amino acids, with from about 5 to about 20 amino acids being preferred, and from about 7 to about 15 being particularly preferred. The peptides can be digests of naturally occurring proteins, random peptides, or “biased” random peptides. In some methods, the test agents are polypeptides or proteins.

The test agents can also be nucleic acids. Nucleic acid test agents can be naturally occurring nucleic acids, random nucleic acids, or “biased” random nucleic acids. For example, digests of prokaryotic or eukaryotic genomes can be similarly used as described above for proteins.

In some preferred methods, the test agents are small molecules (e.g., molecules with a molecular weight of not more than about 1,000). Preferably, high throughput assays are adapted and used to screen for such small molecules. In some methods, combinatorial libraries of small molecule test agents as described above can be readily employed to screen for small molecule modulators of TRAILs. A number of assays are available for such screening, e.g., as described in Schultz (1998) Bioorg Med Chem Lett 8:2409-2414; Weller (1997) Mol Divers. 3:61-70; Fernandes (1998) Curr Opin Chem Biol 2:597-603; and Sittampalam (1997) Curr Opin Chem Biol 1:384-91.

Libraries of test agents to be screened with the claimed methods can also be generated based on structural studies of the apoptosis-modulatory polypeptides, their fragments or analogs. Such structural studies allow the identification of test agents that are more likely to bind to the apoptosis-modulatory polypeptides. The three-dimensional structure of an apoptosis-modulatory polypeptide can be studied in a number of ways, e.g., crystal structure and molecular modeling. Methods of studying protein structures using x-ray crystallography are well known in the literature. See Physical Bio-chemistry, Van Holde, K. E. (Prentice-Hall, New Jersey 1971), pp. 221-239, and Physical Chemistry with Applications to the Life Sciences, D. Eisenberg & D. C. Crothers (Benjamin Cummings, Menlo Park 1979). Computer modeling of apoptosis-modulatory polypeptides' structures provides another means for designing test agents for screening modulators of TRAIL-induced apoptosis. Methods of molecular modeling have been described in the literature, e.g., U.S. Pat. No. 5,612,894 entitled “System and method for molecular modeling utilizing a sensitivity factor”, and U.S. Pat. No. 5,583,973 entitled “Molecular modeling method and system”. In addition, protein structures can also be determined by neutron diffraction and nuclear magnetic resonance (NMR). See, e.g., Physical Chemistry, 4th Ed. Moore, W. J. (Prentice-Hall, New Jersey 1972), and NMR of Proteins and Nucleic Acids, K. Wuthrich (Wiley-Interscience, New York 1986).

Modulators of the present invention also include antibodies that specifically bind to an apoptosis-modulatory polypeptide in Tables 1 and 2. Such antibodies can be monoclonal or polyclonal. Such antibodies can be generated using methods well known in the art. For example, the production of non-human monoclonal antibodies, e.g., murine or rat, can be accomplished by, for example, immunizing the animal with an apoptosis-modulatory polypeptide or its fragment (See Harlow & Lane, Antibodies, A Laboratory Manual, Cold Spring Harbor Laboratory Press, Cold Spring Harbor N.Y.). Such an immunogen can be obtained from a natural source, by peptides synthesis or by recombinant expression.

Humanized forms of mouse antibodies can be generated by linking the CDR regions of non-human antibodies to human constant regions by recombinant DNA techniques. See Queen et al., Proc. Natl. Acad. Sci. USA 86, 10029-10033 (1989) and WO 90/07861. Human antibodies can be obtained using phage-display methods. See, e.g., Dower et al., WO 91/17271; McCafferty et al., WO 92/01047. In these methods, libraries of phage are produced in which members display different antibodies on their outer surfaces. Antibodies are usually displayed as Fv or Fab fragments. Phage displaying antibodies with a desired specificity are selected by affinity enrichment to an apoptosis-modulatory polypeptide of the present invention.

Human antibodies against an apoptosis-modulatory polypeptide can also be produced from non-human transgenic mammals having transgenes encoding at least a segment of the human immunoglobulin locus and an inactivated endogenous immunoglobulin locus. See, e.g., Lonberg et al., WO93/12227 (1993); Kucherlapati, WO 91/10741 (1991). Human antibodies can be selected by competitive binding experiments, or otherwise, to have the same epitope specificity as a particular mouse antibody. Such antibodies are particularly likely to share the useful functional properties of the mouse antibodies. Human polyclonal antibodies can also be provided in the form of serum from humans immunized with an immunogenic agent. Optionally, such polyclonal antibodies can be concentrated by affinity purification using an apoptosis-modulatory polypeptide or its fragment.

C. Screening Test Agents that Modulate Apoptosis-Modulatory Polypeptides

A number of assay systems can be employed to screen test agents for modulators of an apoptosis-modulatory polypeptide. As noted above, the screening can utilize an in vitro assay system or a cell-based assay system. In this screening step, test agents can be screened for binding to the apoptosis-modulatory polypeptide, altering cellular level of the apoptosis-modulatory polypeptide, or modulating other biological activities of the apoptosis-modulatory polypeptide.

A variety of routinely practiced assays can be used to identify test agents that modulate an apoptosis-modulatory polypeptide. Preferably, the test agents are screened with a cell based assay system. For example, in a typical cell based assay for screening test agents for modulators of expression of an apoptosis-modulatory polypeptide, a construct comprising a transcription regulatory element of the apoptosis-modulatory polypeptide that is operably linked to a reporter gene is introduced into a host cell system. The reporter gene activity (e.g., an enzymatic activity) in the presence of a test agent can be determined and compared to the activity of the reporter gene in the absence of the test agent. An increase or decrease in the activity identifies a modulating agent the apoptosis-modulatory polypeptide. The reporter gene can encode any detectable polypeptide (response or reporter polypeptide) known in the art, e.g., detectable by fluorescence or phosphorescence or by virtue of its possessing an enzymatic activity. The detectable response polypeptide can be, e.g., luciferase, alpha-glucuronidase, alpha-galactosidase, chloramphenicol acetyl transferase, green fluorescent protein, enhanced green fluorescent protein, and the human secreted alkaline phosphatase.

In the cell-based assays, the test agent (e.g., a peptide or a polypeptide) can also be expressed from a different vector that is also present in the host cell. In some methods, a library of test agents are encoded by a library of such vectors. Such libraries can be generated using methods well known in the art (see, e.g., Sambrook et al. and Ausubel et al., supra) or obtained from a variety of commercial sources.

In addition to cell based assays described above, modulating agents of an apoptosis-modulatory polypeptide can also be screened with non-cell based methods. These methods include, e.g., mobility shift DNA-binding assays, methylation and uracil interference assays, DNase and hydroxy radical footprinting analysis, fluorescence polarization, and UV crosslinking or chemical cross-linkers. For a general overview, see, e.g., Ausubel et al., supra (chapter 12, DNA-Protein Interactions). One technique for isolating co-associating proteins, including nucleic acid and DNA/RNA binding proteins, includes use of UV crosslinking or chemical cross-linkers, including e.g., cleavable cross-linkers dithiobis (succinimidylpropionate) and 3,3′-dithiobis (sulfosuccinimidyl-propionate); see, e.g., McLaughlin (1996) Am. J. Hum. Genet. 59:561-569; Tang (1996) Biochemistry 35:8216-8225; Lingner (1996) Proc. Natl. Acad. Sci. USA 93:10712; Chodosh (1986) Mol. Cell. Biol 6:4723-4733.

In some methods, binding of a test agent to an apoptosis-modulatory polypeptide is determined in the first screening step. Binding of test agents to an apoptosis-modulatory polypeptide can be assayed by a number of methods including e.g., labeled in vitro protein-protein binding assays, electrophoretic mobility shift assays, immunoassays for protein binding, and functional assays (phosphorylation assays, etc.). See, e.g., U.S. Pat. Nos. 4,366,241; 4,376,110; 4,517,288; and 4,837,168; and also Bevan et al., Trends in Biotechnology 13:115-122, 1995; Ecker et al., Bio/Technology 13:351-360, 1995; and Hodgson, Bio/Technology 10:973-980, 1992. The test agent can be identified by detecting a direct binding to the apoptosis-modulatory polypeptide, e.g., co-immunoprecipitation with the apoptosis-modulatory polypeptide by an antibody directed to the apoptosis-modulatory polypeptide. The test agent can also be identified by detecting a signal that indicates that the agent binds to the apoptosis-modulatory polypeptide, e.g., fluorescence quenching.

Competition assays provide a suitable format for identifying test agents that specifically bind to an apoptosis-modulatory polypeptide. In such formats, test agents are screened in competition with a compound already known to bind to the apoptosis-modulatory polypeptide. The known binding compound can be a synthetic compound. It can also be an antibody, which specifically recognizes the apoptosis-modulatory polypeptide, e.g., a monoclonal antibody directed against the apoptosis-modulatory polypeptide. If the test agent inhibits binding of the compound known to bind the apoptosis-modulatory polypeptide, then the test agent also binds the apoptosis-modulatory polypeptide.

Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242-253 (1983)); solid phase direct biotin-avidin EIA (see Kirkland et al., J. Immunol. 137:3614-3619 (1986)); solid phase direct labeled assay, solid phase direct labeled sandwich assay (see Harlow and Lane, “Antibodies, A Laboratory Manual,” Cold Spring Harbor Press (1988)); solid phase direct label RIA using ¹²⁵I label (see Morel et al., Mol. Immunol. 25(1):7-15 (1988)); solid phase direct biotin-avidin EIA (Cheung et al., Virology 176:546-552 (1990)); and direct labeled RIA (Moldenhauer et al., Scand. J. Immunol. 32:77-82 (1990)). Typically, such an assay involves the use of purified polypeptide bound to a solid surface or cells bearing either of these, an unlabelled test agent and a labeled reference compound. Competitive inhibition is measured by determining the amount of label bound to the solid surface or cells in the presence of the test agent. Usually the test agent is present in excess. Modulating agents identified by competition assay include agents binding to the same epitope as the reference compound and agents binding to an adjacent epitope sufficiently proximal to the epitope bound by the reference compound for steric hindrance to occur. Usually, when a competing agent is present in excess, it will inhibit specific binding of a reference compound to a common target polypeptide by at least 50 or 75%.

The screening assays can be either in insoluble or soluble formats. One example of the insoluble assays is to immobilize an apoptosis-modulatory polypeptide or its fragments onto a solid phase matrix. The solid phase matrix is then put in contact with test agents, for an interval sufficient to allow the test agents to bind. After washing away any unbound material from the solid phase matrix, the presence of the agent bound to the solid phase allows identification of the agent. The methods can further include the step of eluting the bound agent from the solid phase matrix, thereby isolating the agent. Alternatively, other than immobilizing the apoptosis-modulatory polypeptide, the test agents are bound to the solid matrix and the apoptosis-modulatory polypeptide molecule is then added.

Soluble assays include some of the combinatory libraries screening methods described above. Under the soluble assay formats, neither the test agents nor the apoptosis-modulatory polypeptide are bound to a solid support. Binding of an apoptosis-modulatory polypeptide or fragment thereof to a test agent can be determined by, e.g., changes in fluorescence of either the apoptosis-modulatory polypeptide or the test agents, or both. Fluorescence may be intrinsic or conferred by labeling either component with a fluorophor.

In some binding assays, either the apoptosis-modulatory polypeptide, the test agent, or a third molecule (e.g., an antibody against the apoptosis-modulatory polypeptide) can be provided as labeled entities, i.e., covalently attached or linked to a detectable label or group, or cross-linkable group, to facilitate identification, detection and quantification of the polypeptide in a given situation. These detectable groups can comprise a detectable polypeptide group, e.g., an assayable enzyme or antibody epitope. Alternatively, the detectable group can be selected from a variety of other detectable groups or labels, such as radiolabels (e.g., ¹²⁵I, ³²P, ³⁵S) or a chemiluminescent or fluorescent group. Similarly, the detectable group can be a substrate, cofactor, inhibitor or affinity ligand.

Binding of a test agent to an apoptosis-modulatory polypeptide provides an indication that the agent can be a modulator of the apoptosis-modulatory polypeptide. It also suggests that the agent may modulate TRAIL bioactivities (e.g., by binding to and modulate the apoptosis-modulatory polypeptide which in turn acts on TRAIL-induced apoptosis). Thus, a test agent that binds to an apoptosis-modulatory polypeptide can be further tested for ability to modulate TRAIL-induced apoptosis (i.e., in the second testing step outlined above). Alternatively, a test agent that binds to an apoptosis-modulatory polypeptide can be further examined to determine its activity on the apoptosis-modulatory polypeptide. The existence, nature, and extent of such activity can be tested by an activity assay. Such an activity assay can confirm that the test agent binding to the apoptosis-modulatory polypeptide indeed has a modulatory activity on the apoptosis-modulatory polypeptide.

More often, activity assays can be used independently to identify test agents that modulate activities of an apoptosis-modulatory polypeptide (i.e., without first assaying their ability to bind to the apoptosis-modulatory polypeptide). In general, such methods involve adding a test agent to a sample containing an apoptosis-modulatory polypeptide in the presence or absence of other molecules or reagents which are necessary to test a biological activity of the apoptosis-modulatory polypeptide (e.g., kinase activity if the apoptosis-modulatory polypeptide is a kinase), and determining an alteration in the biological activity of the apoptosis-modulatory polypeptide. In addition to assays for screening agents that modulate an enzymatic or other biological activities of an apoptosis-modulatory polypeptide, the activity assays also encompass in vitro screening and in vivo screening for alterations in expression or cellular level of the apoptosis-modulatory polypeptide.

In an exemplary embodiment, the apoptosis-modulatory polypeptide is a kinase, and the test agent is examined for ability to modulate the kinase activity of the apoptosis-modulatory polypeptide. For example, the kinase activity of JIK can be assayed as described in the art, e.g., Tassi et al., J Biol Chem 274: 33287-95, 1999. When Gsk3α is used in the screening, its kinase activity can be assayed as described in Embi et al., Eur. J. Biochem. 107: 519-527, 1980; Welsh et al., Biochem. J. 294: 625-629, 1993; and Nikoulina et al., Diabetes 51(7):2190-8, 2002. When other apoptosis-modulatory polypeptides are employed, e.g., SRP72, their biological activities can be similarly assayed using methods disclosed in the art. For example, protein translocation activity of SRP72 can be examined with methods described in, e.g., ER transport assay using microsomes and in vitro translated polypeptide (Utz et al., J Biol Chem 273: 35362-70, 1998; and Fehrmann et al., J Virol 77(11): 6293-304, 2003).

D. Screening for Agents that Modulate TRAIL-Induced Apoptosis

Once a modulating agent has been identified to bind to an apoptosis-modulatory polypeptide and/or to modulate a biological activity (including cellular level) of the apoptosis-modulatory polypeptide, it can be further tested for ability to modulate TRAIL-induced apoptosis. Modulation of TRAIL-induced apoptosis by the modulating agent is typically tested in the presence of the apoptosis-modulatory polypeptide. Typically, to examine apoptotic activity of a cell, the apoptosis-modulatory polypeptide is endogenously expressed in the cell.

The modulating agents screened in the first assay step can either positively or negatively modulate apoptosis-modulatory polypeptides. As noted above, the apoptosis-modulatory polypeptides identified by the prensent inventors either inhibit or enhance TRAIL-induced apoptosis. If an apoptosis-enhancing polypeptide is employed in the sreening (e.g., FLJ32312 (DOBI), Gsk3α and SRP72), a modulating agent that positively modulate the apoptosis-modulatory polypeptide, e.g., upregulates its cellular level or biological activities, is likely to be a potential stimulator of TRAIL-induced apoptosis. Conversely, a modulating agent that down-regulates cellular level or other activities of the apoptosis-modulatory polypeptide is a potential inhibitor of TRAIL-induced apoptosis. On the other hand, if an apoptosis-inhibitory polypeptide is employed in the screening (e.g., FLJ21802 (MIRSA), JIK, and PLXNB1), a modulating agent that positively modulates the apoptosis-modulatory polypeptide would be a candidate for inhibitor of TRAIL-induced apoptosis. Conversely, a modulating agent that down-regulates the apoptosis-modulatory polypeptide makes a potential stimulator of TRAIL-induced apoptosis.

Various assays for analyzing apoptosis have been described in the art and can be readily employed to screen for test agents that modulate TRAIL-induced apoptosis activities. In some embodiments, an apoptosis assay can be employed to monitor effects of modulating agents identified in the first screening step on TRAIL-mediated apoptosis. This assay is well known and routinely practiced in the art (see, e.g., Lichtenstein et al., J Virol 76: 11329-42, 2002). TRAIL-induced apoptosis can also be monitored using a DNA fragmentation assay. This assay can be performed as described in the art, e.g., Sellins et al., J. Immunol. 139: 3199-206, 1987; and Sah et al., J Biol Chem 2003 Mar 27; epub ahead of print. In other embodiments, effects of modulating agents on TRAIL-induced apoptosis can be examined as described in the Examples below, e.g., assaying TRAIL-dependent caspase activation or cellular death.

With any of these assays (e.g., a caspase assay or cellular death assay), to examine whether a modulating agent identified in the first screen step is indeed a modulator of TRAIL-induced apoptosis, the modulating agent is applied to the cells to be tested for apoptosis activity (e.g., Hela cells or colon cancer line HCT15). The assay is performed in the presence or absence of TRAIL. If TRAIL-dependent apoptosis activity is altered by the addition of the test agent to the assay, the modulating agent identified in the first screen step is confirmed as a modulator of TRAIL-induced apoptosis.

Some of the apoptosis-modulatory polypeptides (e.g., MIRSA and JIK) also modulate TRAIL-independent apoptosis, as detailed in the Examples. When these targets are employed in the screening, the modulating agents identified in the first step can be examined for effects on apoptosis in the absence of TRAIL.

IV. Therapeutic Applications

The present invention provides novel methods and compositions for modulating apoptotic activities of cells. The methods and compositions of the present invention find therapeutic applications in treating various clinical conditions or disease states that are linked to abnormal cell proliferation, e.g., various forms of cancer. Modulation of TRAIL-induced apoptosis activities is also useful for preventing or modulating the development of such diseases or disorders in a subject suspected of being, or known to be, prone to such diseases or disorders.

To modulate apoptotic activities of cells, the cells can be contacted with any a number of the modulators identified in accordance with the present invention. In some methods, a modulator of TRAIL of the present invention is introduced directly to a subject (e.g., a human, a mammal, or other non-human animal). The novel modulators of TRAIL-induced apoptosis can also be used in combination with other known agents in modulating apoptosis. This could lead to synergistic effect in modulating (e.g., inducing) apoptotic activities of the cells. Typically, the other known agents are agonists or activators of apoptosis or antagonists of inhibitors of apoptosis. For example, modulating agents that promote TRAIL-induced apoptosis can be administered to a subject together with a TRAIL polypeptide to treat caner in the subject by promoting TRAIL-induced apoptosis. The TRAIL polypeptide is described in U.S. Pat. Nos. 5,763,223 and 6,284,236. Methods of using TRAIL to treat tumors have been disclosed in the art, e.g., in U.S. Pat. No. 6,284,236; and U.S. Patent Application Publication No. 20020169123 (Nov. 14, 2002).

A. Examples of Disease and Conditions Amenable to Treatment

A great number of diseases and conditions are amenable to treatment with methods and compositions of the present invention. Examples of tumors that can be treated with methods and compositions of the present invention include but are not limited to skin, breast, brain, cervical carcinomas, testicular carcinomas. They encompass both solid tumors or metastatic tumors. Cancers that can be treated by the compositions and methods of the invention include cardiac cancer (e.g., sarcoma, myxoma, rhabdomyoma, fibroma, lipoma and teratoma); lung cancer (e.g., bronchogenic carcinoma, alveolar carcinoma, bronchial adenoma, sarcoma, lymphoma, chondromatous hamartoma, mesothelioma); various gastrointestinal cancer (e.g., cancers of esophagus, stomach, pancreas, colon, small bowel, and large bowel); genitourinary tract cancer (e.g., kidney, bladder and urethra, prostate, testis; liver cancer (e.g., hepatoma, cholangiocarcinoma, hepatoblastoma, angiosarcoma, hepatocellular adenoma, hemangioma); bone cancer (e.g., osteogenic sarcoma, fibrosarcoma, malignant fibrous histiocytoma, chondrosarcoma, Ewing's sarcoma, malignant lymphoma, multiple myeloma, malignant giant cell tumor chordoma, osteochronfroma, benign chondroma, chondroblastoma, chondromyxofibroma, osteoid osteoma and giant cell tumors); cancers of the nervous system (e.g., of the skull, meninges, brain, and spinal cord); gynecological cancers (e.g., uterus, cervix, ovaries, vulva, vagina); hematologic cancer (e.g., cancers relating to blood, Hodgkin's disease, non-Hodgkin's lymphoma); skin cancer (e.g., malignant melanoma, basal cell carcinoma, squamous cell carcinoma, Karposi's sarcoma, moles dysplastic nevi, lipoma, angioma, dermatofibroma, keloids, psoriasis); and cancers of the adrenal glands (e.g., neuroblastoma).

Disease states other than cancer may also be treated by the methods and compositions of the invention. These include restenosis, autoimmune disease, arthritis, graft rejection, inflammatory bowel disease, proliferation induced after medical procedures such as surgery, angioplasty, and the like. In some other applications, modulators that inhibit TRAIL-induced apoptosis can be used to reduce apoptosis where and when it is detrimental. One such disorder is thrombotic thrombocytopenic purpura (TTP) (Thompson et al., Blood 80:1890, 1992; and Torok et al., Am. J. Hematol. 50:84, 1995). Another thrombotic microangiopathy is hemolytic-uremic syndrome (HUS) (Moake et al., Lancet, 343: 393, 1994; Melnyk et al., Arch. Intern. Med. 155: 2077, 1995), which is also amenable to treatment with the compositions of the present inveniton.

B. Pharmaceutical Compositions and Administration

The apoptosis modulators of the present invention can be directly administered under sterile conditions to the subject to be treated. The modulators can be administered alone or as the active ingredient of a pharmaceutical composition. Therapeutic composition of the present invention can be combined with or used in association with other therapeutic agents. For example, a subject may be treated with a pharmaceutical composition comprising an effective amount of a TRAIL polypeptide and one novel modulator of the present invention that modulates TRAIL-induced apoptosus. A subjet with tumor or cancer can also be treated simulataneously with conventional chemotherapeutic agents. Such chemotherapeutic agents are well known in the art, e.g., daunorubicin or epirubicin. See, generally, The Merck Manual of Diagnosis and Therapy, 15th Ed., pp. 1206-1228, Berkow et al., eds., Rahay, N.J., 1987). When used with the modulators of the invention, such chemotherapeutic agents may be used individually, sequentially, or in combination with one or more other such chemotherapeutic agents.

Pharmaceutical compositions of the present invention typically comprise at least one active ingredient together with one or more acceptable carriers thereof. Pharmaceutically carriers enhance or stabilize the composition, or to facilitate preparation of the composition. Pharmaceutically acceptable carriers are determined in part by the particular composition being administered (e.g., nucleic acid, protein, modulatory compounds or transduced cell), as well as by the particular method used to administer the composition. They should also be both pharmaceutically and physiologically acceptable in the sense of being compatible with the other ingredients and not injurious to the subject. This carrier may take a wide variety of forms depending on the form of preparation desired for administration, e.g., oral, sublingual, rectal, nasal, or parenteral. For example, the apoptosis modulator can be complexed with carrier proteins such as ovalbumin or serum albumin prior to their administration in order to enhance stability or pharmacological properties.

There are a wide variety of suitable formulations of pharmaceutical compositions of the present invention (see, e.g., Remington: The Science and Practice of Pharmacy, Mack Publishing Co., 20^{th} ed., 2000). Without limitation, they include syrup, water, isotonic saline solution, 5% dextrose in water or buffered sodium or ammonium acetate solution, oils, glycerin, alcohols, flavoring agents, preservatives, coloring agents starches, sugars, diluents, granulating agents, lubricants, and binders, among others. The carrier may also include a sustained release material such as glyceryl monostearate or glyceryl distearate, alone or with a wax.

The pharmaceutical compositions can be prepared in various forms, such as granules, tablets, pills, suppositories, capsules, suspensions, salves, lotions and the like. The concentration of therapeutically active compound in the formulation may vary from about 0.1-100% by weight. Therapeutic formulations are prepared by any methods well known in the art of pharmacy. See, e.g., Gilman et al., eds., Goodman and Gilman's: The Pharmacological Bases of Therapeutics, 8th ed., Pergamon Press, 1990; Remington: The Science and Practice of Pharmacy, Mack Publishing Co., 20^{th} ed., 2000; Avis et al., eds., Pharmaceutical Dosage Forms: Parenteral Medications, published by Marcel Dekker, Inc., N.Y., 1993; Lieberman et al., eds., Pharmaceutical Dosage Forms: Tablets, published by Marcel Dekker, Inc., N.Y., 1990; and Lieberman et al., eds., Pharmaceutical Dosage Forms: Disperse Systems, published by Marcel Dekker, Inc., N.Y., 1990.

The therapeutic formulations can be delivered by any effective means that could be used for treatment. Depending on the specific apoptosis modulators to be administered, the suitable means include oral, rectal, vaginal, nasal, pulmonary administration, or parenteral (including subcutaneous, intramuscular, intravenous and intradermal) infusion into the bloodstream. For parenteral administration, apoptosis modulators (including polynucleotides encoding apoptosis modulators) of the present invention may be formulated in a variety of ways. Aqueous solutions of the modulators may be encapsulated in polymeric beads, liposomes, nanoparticles or other injectable depot formulations known to those of skill in the art. The nucleic acids may also be encapsulated in a viral coat. Additionally, the compounds of the present invention may also be administered encapsulated in liposomes. The compositions, depending upon its solubility, may be present both in the aqueous layer and in the lipidic layer, or in what is generally termed a liposomic suspension. The hydrophobic layer, generally but not exclusively, comprises phospholipids such as lecithin and sphingomyelin, steroids such as cholesterol, more or less ionic surfactants such a diacetylphosphate, stearylamine, or phosphatidic acid, and/or other materials of a hydrophobic nature.

The compositions may be supplemented by active pharmaceutical ingredients, where desired. Optional antibacterial, antiseptic, and antioxidant agents may also be present in the compositions where they will perform their ordinary functions.

The therapeutic formulations can conveniently be presented in unit dosage form and administered in a suitable therapeutic dose. A suitable therapeutic dose can be determined by any of the well known methods such as clinical studies on mammalian species to determine maximum tolerable dose and on normal human subjects to determine safe dosage. Except under certain circumstances when higher dosages may be required, the preferred dosage of an apoptosis modulator usually lies within the range of from about 0.001 to about 1000 mg, more usually from about 0.01 to about 500 mg per day.

The preferred dosage and mode of administration of an apoptosis modulator can vary for different subjects, depending upon factors that can be individually reviewed by the treating physician, such as the condition or conditions to be treated, the choice of composition to be administered, including the particular apoptosis modulator, the age, weight, and response of the individual subject, the severity of the subject's symptoms, and the chosen route of administration. As a general rule, the quantity of an apoptosis modulator administered is the smallest dosage that effectively and reliably prevents or minimizes the conditions of the subjects. Therefore, the above dosage ranges are intended to provide general guidance and support for the teachings herein, but are not intended to limit the scope of the invention.

In some applications, a polynucleotide encoding a modulator of TRAIL of the present invention is introduced by retroviral or other means. For example, polynucleotides with sequences encoding the novel modulators of TRAIL-induced apoptosis or the TRAIL-polypeptides of the present invention, or substantially identical sequences or their fragments, can be used to modulate apoptotic activities of cells. In some methods, therapeutic compositions comprising the polynucleotides are transfected into cells for therapeutic purposes in vitro and in vivo. These polynucleotides can be inserted into any of a number of well-known vectors for the transfection of target cells and organisms as described below. The nucleic acids are transfected into cells, ex vivo or in vivo, through the interaction of the vector and the target cell. The compositions are administered to a subject in an amount sufficient to elicit a therapeutic response in the subject.

Such gene therapy procedures have been used to correct acquired and inherited genetic defects, cancer, and viral infection in a number of contexts. The ability to express artificial genes in humans facilitates the prevention and/or cure of many important human diseases, including many diseases which are not amenable to treatment by other therapies (for a review of gene therapy procedures, see Anderson, Science 256:808-813 (1992); Nabel & Felgner, TIBTECH 11:211-217 (1993); Mitani & Caskey, TIBTECH 11: 162-166 (1993); Mulligan, Science 926-932 (1993); Dillon, TIBTECH 11:167-175 (1993); Miller, Nature 357:455-460 (1992); Van Brunt, Biotechnology 6(10):1149-1154 (1998); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology (Doerfler & Böhm eds., 1995); and Yu et al., Gene Therapy 1:13-26 (1994)).

### EXAMPLES

The following examples are provided to illustrate, but not to limit the present invention.

### Example 1

### General Methods

Tissue Culture: Hela cells were cultured in DMEM supplemented with 10% FBS, penicillin, streptomycin, L-glutamine and 1% nonessential amino acids (Invitrogen, Carlsbad, Calif.), at 37° C. with 95% CO₂/5% O₂.

SiRNA Library Preparation and High-Throughput Transfection: siRNAs were purchased from Dharmacon (Lafayette, Colo.) as single stranded RNA oligos, and then annealed and prepared in 96 well plates according to manufacturer instructions. The whole collection was normalized to a final concentration of 8 ng/μl in 1100 mM KOAc, 30 mM HEPES-KOH, 2 mM MgOAc, pH 7.4. 1 μl of each siRNA was then transferred to 384 well plates (Greiner, Longwood, Fla.) in duplicate, and stored at −80C.

384-well microplate reverse transfections: Lipofectamine2000 (Invitrogen) was diluted with Opti-MeM (Gibco, Gaithersburg, Md.) and then added to 384 well siRNA library plates using a Titertech 96/384 microplate liquid dispenser. HeLa cells were then seeded at 4000 cells/384 well using the Titertech dispenser, and incubated for 48 hrs prior to treatment with TRAIL. Under these conditions, cells transfected with control siRNAs reached approximately 85% confluence prior to the treatment.

Cellular death assay: Treatment without or with 1 mg/ml TRAIL (CalbioChem, San Diego, Calif.) were carried out for an additional 24 hour period in 1% serum conditions to increase TRAIL sensitivity, followed by viability measurement using 10% AlamarBlue (Trek Diagnostic, Cleveland, Ohio).

Caspase 3/Caspase7 Assay: HeLa cells were transfected in duplicate wells and treated with TRAIL ligand as described above. Following a 48 hour incubation period, enhancers in the screen were treated for 4-6 hours at a final concentration of TRAIL at 0.1 μg/ml. Inhibitors in the screen were treated for 20 h at a final concentration of TRAIL at 1 μg/ml. After desired time of treatment, Caspase 3/7 activity was determined using a fluorometric assay (Apo-One Caspase 3/7 kit, Promega Madison, Wis.). Treatments here were carried in 10% serum conditions to assure that screening results are not dependent on low serum conditions. Readings were taken on a SpectraMax Pro (Molecular Devices, Sunnyvale, Calif.) and normalized against samples transfected with siG12 control siRNA treated with 1 μg/ml of TRAIL.

Western Blots: HeLa cells were transfected with siRNA oligos under our standard Lipofectamine 2000 conditions. Cell lysates were collected from 12 well format approximately 48 hours post-transfection using a hypotonic lysis buffer (Hepes pH 7.2 20 mM, MgCl₂ 1.5 mM, KCl 10 mM, EDTA 1 mM, 1% Triton X-100). Lysates were then quantified for total protein levels using BCA Protein Assay (Pierce, Rockford, Ill.) and equal amounts loaded into Novex Tris-Gly gels (Invitrogen). Proteins were transferred onto PVDF membranes with standard Novex TBS-Tween transfer buffers/Blot module (Invitrogen). Antibodies of GSK 3α/β (Stressgen, San Diego, Calif.), Caspase-3 (Pharmingen, San Diego, Calif.), Caspase-8 (Pharmingen), Capsase-9 (MBL, Watertown, Mass.), BID (Pharmingen) and Actin (Santa Cruz Biotechnology, Santa Cruz, Calif.) were used for primary blotting as suggested by manufacture datasheet. Specific secondary antibodies conjugated to HRP were obtained from BiORad (Hercules, Calif.) and used at suggested concentrations. Quantification of protein within membranes was performed using ECL-Plus Western Blot Detection System (Amersham Biosystems, Piscattaway, N.J.). Membranes were exposed using Hyperfilm ECL (Amersham Pharmacia).

### Examples 2

### siRNA Screening Strategy

Target knockdown efficiency and specificity are the primary factors that make RNAi a preferred screening method. However, efficacy varies among siRNAs for a given target and the identification of optimal siRNAs remains largely a matter of trial and error. For our initial siRNA library we chose to focus on kinases, a particularly active class of proteins that are involved in variety of cellular phenomena. Since many kinases are highly related, this library also allows us to determine the individual contribution among highly similar proteins to a given biological process. In total, the siRNA library targeted 510 genes which included 380 known and predicted kinases, 20 known genes of interest, 100 genes of unknown function and 10 well characterized genes known to play a role in apoptosis and TRAIL-mediated signaling pathways. Based on previous results, we anticipate that approximately 2/3 of these siRNAs would lead to significant target knockdown (70% reduction or more), although the actual inhibition required to detect a cellular phenotype in a given assay may vary greatly from one gene to the next. Thus, lack of activity of any given siRNA could be due to i) the target not being expressed or being irrelevant in a given assay, ii) insufficient down-regulation of the target for a phenotype to be observed, or iii) unexpected mismatches among a siRNA and the sequence of its target present in the cell type under study. Since it is not possible to discriminate between these possibilities at this time, only siRNAs inducing a phenotype are informative.

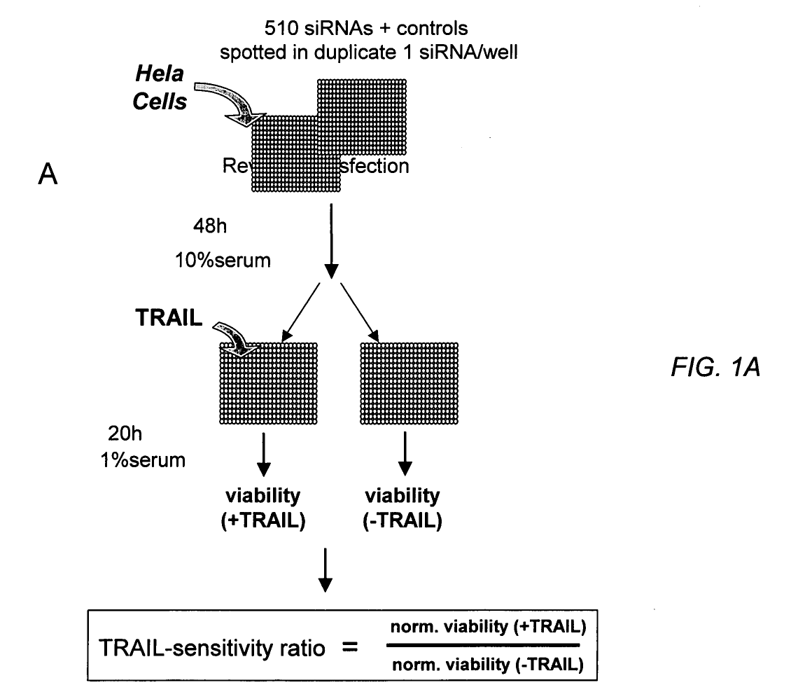

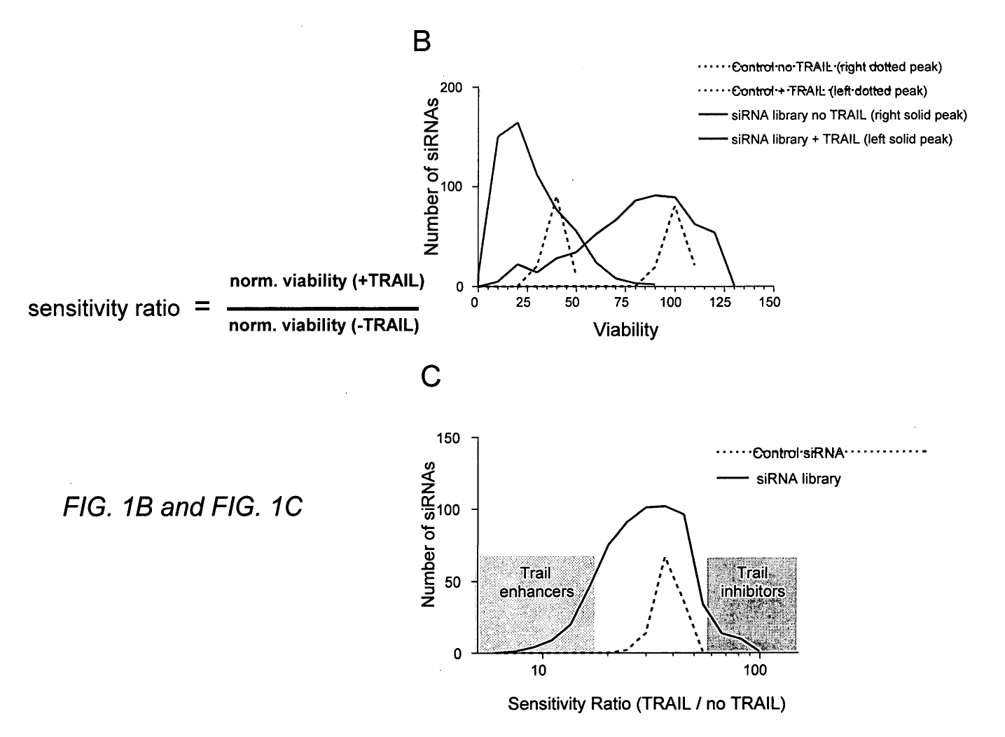

To identify modifiers of cell sensitivity to TRAIL-induced death, we compared the effects of siRNA transfection on cell viability in the presence and absence of TRAIL. Two copies of the siRNA library and relevant controls were transfected into HeLa cells in duplicate and TRAIL was added to one of the library copies for an additional 24 hour period, followed by cell viability measurement (FIG. 1A). Controls included siRNAs against luciferase (negative controls), and siRNAs against various genes involved in apoptosis (positive controls, as described below). The viability of cells transfected with controls decreased from 100% in the absence of TRAIL to 38% following TRAIL treatment (FIG. 1B, hatched lines). In contrast, transfection of the siRNA library resulted in a broad range of viability values (FIG. 1B, solid lines). To determine if siRNAs impact TRAIL-dependant death, we calculated the ratio of viability in the presence versus the absence of TRAIL (TRAIL-sensitivity ratio). Cells transfected with control siRNAs had a TRAIL sensitivity ratio of 38.5% while cells transfected with the siRNA library ranged from 3% (TRAIL sensitizers) to 95% (TRAIL inhibitors) (FIG. 1C). Thus, these screens yielded both putative TRAIL-sensitizers and inhibitors.

### Example 3

### Analysis of Control siRNAs

Analysis of TRAIL-sensitivity ratio scores for positive control siRNAs provided a first assessment of the performance of the screening methodology. SiRNAs against CASP8, the main transducer of receptor-mediated apoptosis, BID and the TRAIL receptor DR4, were among the strongest TRAIL-inhibitors. The strong activity of BID siRNA indicated that TRAIL signaling in these cells requires engagement of the mitochondrial pathway, and thus, that HeLa cells behave as type II cells. Consistent with this notion, an siRNA targeting the CASP9 activator APAF1 also protected cells from TRAIL-induced death. However, a CASP9 siRNA failed to prevent TRAIL-induced death, and subsequent experiments showed that these siRNAs did not effectively reduce CASP9 mRNA levels. An siRNA targeting DR5 was ineffective at blocking TRAIL-mediated apoptosis. This together with the above observation that siRNA-mediated removal of DR4 alone conferred strong protection from TRAIL suggests that DR4 mediates most of the TRAIL signal in these cells. Although HeLa cells behave as p53 deficient, a p53 siRNA strongly inhibited TRAIL activity but only after a dramatic reduction in cell viability (33% survival in the absence of treatment), suggesting a requirement for p53 in both cell survival and apoptosis in these cells. Finally, two siRNAs against the known anti-apoptotic kinases PAKI and AKTI strongly enhanced TRAIL activity.

Exemplary genes targeted by the inhibitor siRNAs and enhancer siRNAs are shown above in Tables 1 and 2, respectively. In the tables, SR(%) refers to survival ratio of the treated cells. P value was determined by T-test comparing values obtained for each siRNA in the 2 screens (4 data points) with the control siRNA population (60 wells/screen).

### Example 4

### Characterization of Genes that Sensitize Cells to TRAIL

In addition to genes involved in the apoptosis pathway, siRNAs against several known genes also inhibited TRAIL-induced apoptosis. These included MYC and the WNT transducer TCF4. Since MYC is a transcriptional target of TCF4, we postulate a mechanism in which VNT signaling induces MYC, and thereby confers susceptibility to TRAIL. Consistent with this, siRNA mediated removal of the WNT transducer DVL2 also prevented TRIAL-induced apoptosis. Other apoptosis-related genes identified in this manner included the Jun N-terminal Kinase 3 (JNK3), which has been reported to have a distinctive pro-apoptotic role among Jun N-terminal kinase family members in neurons, and death-associated-protein 4 (P52^{rlPK}/DAP4), an inhibitor of the PRKR inhibitor p58^{IPK}, which is reported to interact with p53 and the proapoptotic kinase MST-1. Inactivation of DAP4 should result in inhibition of PRKR, which has been reported to result in inhibition of CASP8, FADD, BAD and BAX expression, all of which are required for TRAIL-induced apoptosis. These results indicate that siRNAs are an effective tool to identify novel functions for genes not previously associated with TRAIL signaling.

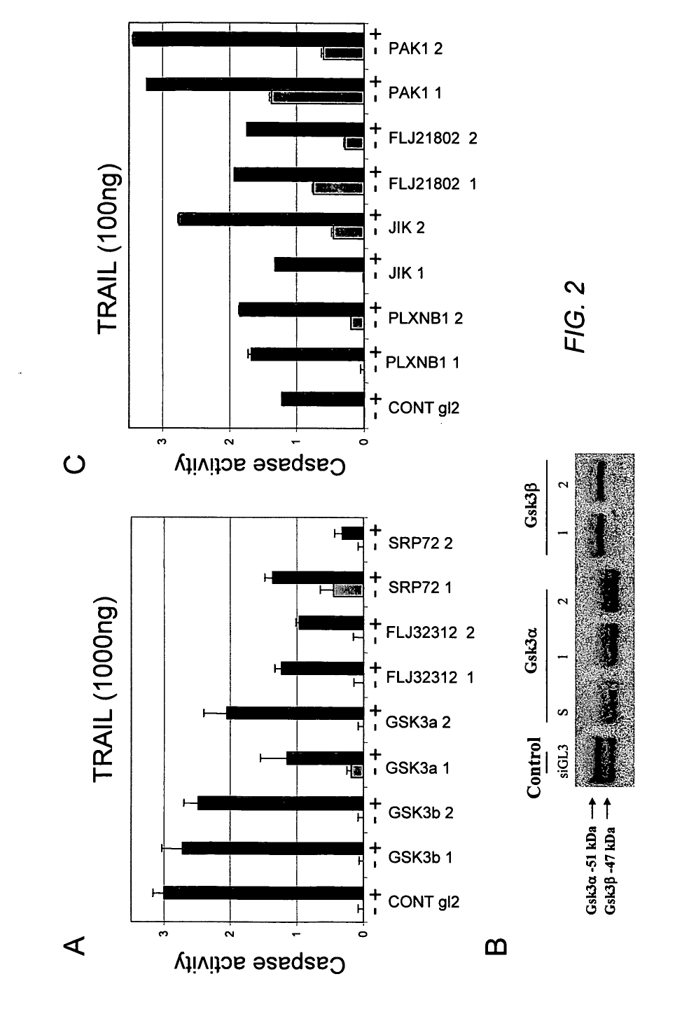

In addition to assigning a role in TRAIL sensitivity to these well studied genes, we also sought to characterize a limited number of TRAIL-inhibitor genes for which no previous role in apoptosis has been defined. Among this class was the kinase GSK3α, the signal recognition particle component and caspase target SRP72, and the hypothetical gene FLJ32312, a novel gene with weak similarity to bacterial pseudo-uridylate synthatses. To ensure that our observations were due to target down-regulation rather than off-target effects or other artifacts, we designed two additional siRNAs against each of these genes and tested their effects on TRAIL-induced cell death. We also included 2 siRNAs against GSK3β to compare them with GSK3α duplexes. To confirm that these siRNAs blocked apoptosis-mediated death we measured caspase activation, the essential process that mediates TRAIL activity. The two additional siRNAs against SRP72, FLJ32312 and GSK3α all inhibited TRAIL-induced caspase activity, confirming the results from the initial screen (FIG. 2A). In contrast, siRNAs targeting GSK3β failed to modulate TRAIL-induced death, suggesting a specific role for GSK3α in modulating TRAIL susceptibility. This was not attributable to suboptimal activity of the GSK3β siRNAs as immunoblot analysis demonstrated that all GSK3α/β siRNAs efficiently and specifically down-regulated the appropriate GSK3 gene product (FIG. 2B). The selective activity of GSK3α in blocking TRAIL-mediated apoptosis is not restricted to HeLa cells since experiments performed in the colon cancer line HCT15 gave similar results.

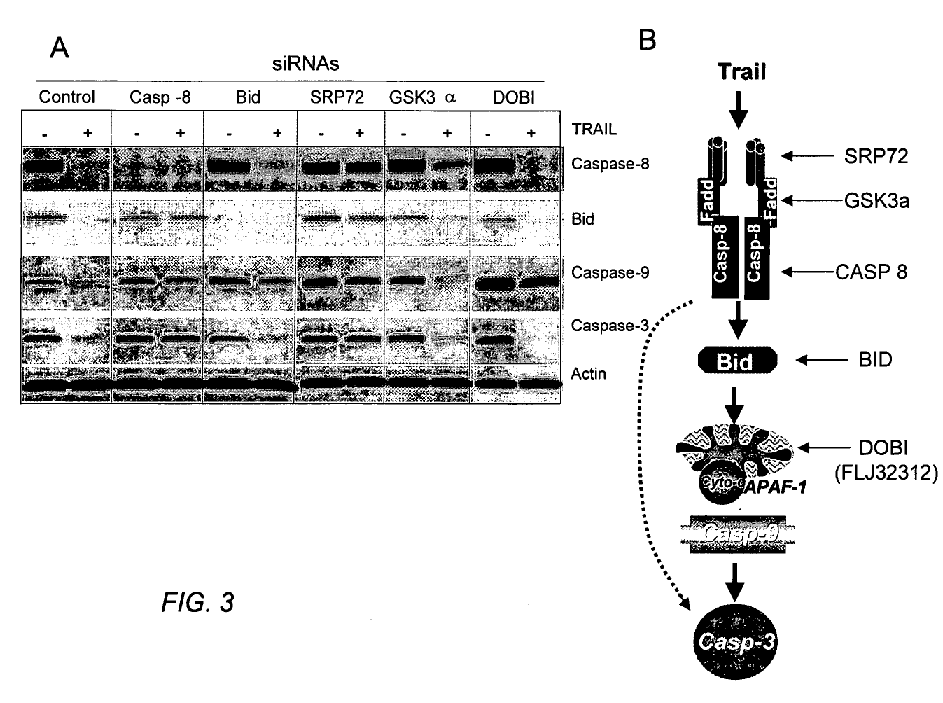

We next sought to determine the level at which these genes were interacting with the TRAIL-induced apoptosis pathway. For this purpose, we performed a biochemical mapping of the caspase cascade by immunoblot analysis of extracts from cells transfected with GSK3α, SRP72 or FLJ32312 siRNAs together with control siRNAs against CASP8 and BID (FIGS. 3A-3B). As expected, the siRNA targeting CASP8 greatly reduced CASP8 protein levels and prevented subsequent downstream signaling events including BID and Caspase 3 processing. Interestingly, the BID siRNA prevented Caspase 9 activation while Caspase 3 was processed normally. This result demonstrates that the intrinsic pathway is required for TRAIL-induced apoptosis in HeLa cells in spite of the direct cleavage of Caspase 3 by Caspase 8. SRP72 silencing resulted in full inhibition of Caspase 8 processing and all subsequent steps in the pathway. The simplest explanation for this observation is that SRP72 functions as an essential component of the signal recognition particle and is required for properly targeting TRAIL receptors or other DISC components to the membrane. GSK3α silencing also resulted in reduction of Caspase 8 processing, although not as markedly as that observed for SRP72. Finally, inhibition of FLJ32312 mimicked BID inhibition by preventing Caspase 9 processing without affecting Caspase 8 or Caspase 3 cleavage. Since BID was normally processed upon TRAIL induction we concluded that FLJ32312 is required for the progression of the apoptotic signal through the mitochondria prior to proteolytic activation of Caspase 9. Activated BID normally causes the release of cytochrome c from the mitochondria to the cytosol where it associates with APAF-1 and CASP9 to form the apoptosome. To determine whether FLJ32312 function was required before or after release of cytochrome c from the mitochondria, we examined cytochrome c concentration in the cytoplasm of cells transfected with siRNA FLJ32312-2, or control siGL3 before and after treatment with TRAIL. As expected, cells transfected with control siRNAs showed a TRAIL-dependent increase in cytosolic cytochrome c levels. In contrast, treatment with siRNA targeting FLJ32312 prevented TRAIL induced cytochrome c release and its subsequent accumulation in the cytoplasmic fraction. These studies suggest that the role of FLJ32312 is to translate BID cleavage into the release of cytochrome c. Sequence analysis of this gene, which we have renamed DOBI (Downstream of BID) showed that this gene is conserved throughout evolution, and contains a conserved consensus ATP/GTP binding motif but failed to reveal similarities to other genes, suggesting that this protein potentially represents a new class of apoptosis modulators.

### Example 5

### Identification of Genes that Limit TRAIL-Induced Death

In addition to identifying TRAIL-sensitizer genes we also sought to identify genes that prevent TRAIL-dependent death. SiRNAs against these genes should enhance cell death in a TRAIL-dependant manner. Many siRNAs in the collection produced a growth disadvantage compared to controls independently of TRAIL (as indicated in FIG. 1B). As expected, the majority of these siRNAs targeted essential cell cycle genes and regulators. To identify siRNAs that selectively increased death in response to TRAIL, we focused on those siRNAs that showed a survival of 70% or greater in the absence of TRAIL, but still accelerated death in the presence of TRAIL. Among the top genes identified, the best characterized were the p38 substrate kinases MKNK1 and MAPKAPK2; RPS6KA5, which plays an anti-apoptotic role through phosphorylation and inactivation of BAD; MEK5 and its specific target BMK1/ERK5, which activates the transcription factor MEF2c; several known anti-apoptotic kinases including AKT1, PAK1, the SRC-family kinases LYN and FGR; and the TCF4-inhibitor nemo-like kinase NLK, underscoring the aforementioned role of TCF4 in TRAIL sensitivity.

Genes that when inhibited by siRNAs increase susceptibility to TRAIL would potentially make attractive therapeutic targets. Therefore, we selected several of the most active sensitizers for further validation and characterization. These were the unknown gene FLJ21802, the JNK inhibitory kinase JIK, and the semaphorin receptor PLXNB1. We also included siRNAs targeting PAK1 as a positive control. As shown in FIG. 2C, transfection of additional siRNAs against these genes resulted in a significant increase in TRAIL-dependent caspase activation with at least one of the duplexes for each target, most obviously with JIK and PAK1 siRNAs. Additionally, we noticed that these siRNAs also induced an increase in TRAIL-independent caspase activation, supporting a more general anti-apoptotic role for these genes. This effect was not observed with siRNAs targeting PLXNB1, and was particularly obvious in the case FLJ21802 and PAK1, which also had also shown an effect on survival during the screen (53% survival in the absence if TRAIL). Sequence analysis of FLJ21802 showed similarity to MINA53, a transcriptional target of MYC with a role in cell proliferation. Because of this sequence similarity and the activity described herein we suggest the more descriptive name Mina53-related suppressor of apoptosis (MIRSA) for this previously uncharacterized gene.

It is understood that the examples and embodiments described herein are for illustrative purposes only and that various modifications or changes in light thereof will be suggested to persons skilled in the art and are to be included within the spirit and purview of this application and scope of the appended claims. Although any methods and materials similar or equivalent to those described herein can be used in the practice or testing of the present invention, the preferred methods and materials are described.

All publications, GenBank sequences, patents and patent applications cited herein are hereby expressly incorporated by reference in their entirety and for all purposes as if each is individually so denoted.

1. A method for identifying agents that modulate TRAIL-induced apoptosis, the method comprising: (a) assaying a biological activity of a polypeptide modulator of TRAIL-induced apoptosis encoded by a gene shown in Tables 1 and 2, or a fragment of said polypeptide, in the presence of test agents to identify one or more modulating agents that modulate the biological activity, and (b) testing one or more of the modulating agents for ability to modulate TRAIL-induced apoptosis; thereby identifying agents that modulate TRAIL-induced apoptosis.

2. The method of claim 1, wherein the polypeptide modulator enhances TRAIL-induced apoptosis and is encoded by a gene shown in Table 1.

3. The method of claim 2, wherein the gene is selected from the group consisting of DOBI, Gsk3α, and SRP72.

4. The method of claim 2, wherein the polypeptide modulator is Gsk3α, and the biological activity is its kinase activity.

5. The method of claim 2, wherein the polypeptide modulator is SRP72, and the biological activity is facilitating protein translocation.

6. The method of claim 1, wherein the polypeptide modulator inhibits TRAIL-induced apoptosis and is encoded by a gene shown in Table 2.

7. The method of claim 6, wherein the gene is selected from the group consisting of MIRSA, JIK, and PLXNB1.

8. The method of claim 6, wherein the polypeptide modulator is JIK, and the biological activity is its kinase activity.

9. The method of claim 6, wherein the polypeptide modulator is PLXNB1, and the biological activity is PLXNB1 binding to semaphorin.

10. The method of claim 1, wherein (a) comprises testing the test agents for ability to bind to the polypeptide modulator.

11. The method of claim 1, wherein (a) comprises testing the test agents for ability to modulate cellular level of the polypeptide modulator.

12. The method of claim 1, wherein (b) comprises testing the modulating agents for ability to modulate caspase activity.

13. The method of claim 1, wherein the assaying of the biological activity of the polypeptide modulator occurs in a cell.

14. A method for modulating TRAIL-induced apoptosis activity of a cell, the method comprising contacting the cell with an effective amount of an agent identified in claim 1, thereby modulating TRAIL-induced apoptosis activity of the cell.

15. The method of claim 14, wherein the agent enhances TRAIL-induced apoptosis activity.

16. The method of claim 14, wherein the cell is a tumor cell.

17. The method of claim 14, wherein the cell is present in a subject.

18. The method of claim 14, wherein the subject is also administered a pharmaceutical composition comprising an effective amount of a TRAIL polypeptide or a fragment thereof.

19. A method for treating cancer in a subject, the method comprising promoting TRAIL-induced apoptosis in the subject by administering to the subject a pharmaceutical composition comprising an effective amount of an agent identified in claim 2, thereby treating cancer in the subject.

20. The method of claim 19, wherein the pharmaceutical composition further comprises an effective amount of a TRAIL polypeptide or a fragment thereof.
